SEQUENCE LISTING (7 sequences)

SEQ ID NO: 1 - protein, 1269 aa, synthetic construct
MNNNGETKCYGNNLDRFIGLREVNKTLRNELIPTDSTRENIEKNGIISEDELRAEMGHELKAIMDDYYRNMIEETLSRKCNINWSTLFAAMDSVARSANKKDADKILEKVQKEKREEIHKLFKEQGNFEKLFKAELVSDVLPDFIGKYEGYSDEERISKLGTIKLFKKFMTSFKAYFDNRKNVFSKENISTSICHRVVNDNAFIFWDNLNAFKKIDKYAHDAVETIEGEISGKLGEWKISQIYSVDFFGMVIPQSGITFYNEVSGEINKYMNIYCQKEKQNSRQFKLKRLHKQILCIGETSFEIPEKYESDEEVYQSVNDFIGMIKNNDIISRLEHLAENVNKYDADKIYINSKSYENVSKCIGGTWNTIKDCLRLWYNENIRGSGKNKEKKVGKAVEEDKYKSIAMLNQLLENYSDGDGRIYNVQLYVEHITKIIGDYKPEELVYDANIRVIEQDEKAEEIKSVLDIYIAILHWIKAFIIEDAVEKDTDFYYEIEEIFDKVQSIIPLYNMVRNYTTQKPYKQEKIKLKFNTPTLAEGWHKSKEYSNNAIILQRDGKYYLGIFNAKNKPDKKIIEGCSQKKNKADYAKMVYYLLPKPHMNLPHIFIKSKEAVKNYGLSNYIVEGYDKDKHIKSSNGFDIKYCRDLIDYFKDCISKHPEWKNFNFSFTDTALYNDIGEFYNEIKRQGYKIEWTYISEEEINRLDENGQIYLFQIYNKDFAEGSKGTPNLHTLYLKNLFSEDNLKDPVFMLNGNTEVFFRKSSIKKPVVHKKGSVLVNRNYIDLNGNRITIPEKEYKEIYEYCNGIGNVNLSESAMKYYDMAKHYTATMDIVKDYRYTVDKFFIHMPMTINFNAESNNNINELALNFVAKNDNIHIIGVDRGERNLIYISVIDSKGNIIKQKSYNVVNDYDYKSKLKEREYDRGDARKNWKRINNIKELKEGYLSQVVHEIAQLVIQYNAIIAMEDLNYGFKRGRFKVERQVYQKFENMLINKLNYLVDKRKNADEAGGVLKGYQLTYIPERLKNMGRQCGIIFYVPAAYTSKIDPTTGFVNLFNFNAITSQEGRRKFINKFKHIRYNLNMNLFEFEFDYNDFITHNTQIAKTEWSVYTNGPRIKKERVNGKWNDGYEADITEVMKQKLSDNGIDYADDGDILTRIAEADDKLVSGIFEIFRLTVQLRNSKSESCGEDYDRIISPVLNESNRFYDSDEYSGENEILPVDADANGAYCIALKGLYEVKQIRENWVEGDKLSGDLLKISNADWFDFIQNKRYE

SEQ ID NO: 2 - DNA, 74 bp, synthetic construct
  source 1..74: mol_type = other DNA
acagacatcatctctgaagtagcgccgccgtatagtgagaaactcactatraggaagagatgatgtctgtagtt

SEQ ID NO: 3 - RNA, 43 nt, synthetic construct
  source 1..43: mol_type = other RNA
taatttctactaagtgtagataactacagacatcatctcttcc

SEQ ID NO: 4 - DNA, 25 bp, synthetic construct
  source 1..25: mol_type = other DNA
ttttttatcgaccgatgctccatag

SEQ ID NO: 5 - RNA, 44 nt, synthetic construct
  source 1..44: mol_type = other RNA
taatttctactaagtgtagatctatggagcatcggtcgataaaa

SEQ ID NO: 6 - DNA, 30 bp, synthetic construct
  source 1..30: mol_type = other DNA
gggggcacgggaggttgcatcgagcggtgg

SEQ ID NO: 7 - RNA, 41 nt, synthetic construct
  source 1..41: mol_type = other RNA
taatttctactaagtgtagatatgcaacctcccgtgccccc

(19) United States
(12) Patent Application Publication
(10) Pub. No.: US 2026/0159875 A1
(43) Pub. Date: Jun. 11, 2026

(54) Cas protein and use thereof in detection

(71) Applicant: NANJING NORMAL UNIVERSITY, Nanjing (CN)

(72) Inventors: Lina WU, Nanjing (CN); He HUANG, Nanjing (CN); Xingxu HUANG, Nanjing (CN); Yanan LI, Nanjing (CN); Shiqi XU, Nanjing (CN); Hairui YU, Nanjing (CN); Yangyang GENG, Nanjing (CN); Jiahui LIU, Nanjing (CN); Weijian SHEN, Nanjing (CN)

(21) Appl. No.: 19/392,209

(22) Filed: Nov. 18, 2025

(30) Foreign Application Priority Data
Dec. 6, 2024 (CN) 202411785623.4

(51) Int. Cl. C12Q 1/6823; C12N 9/22; C12N 15/11; C12Q 1/34; G01N 21/64

(52) U.S. Cl. CPC C12Q 1/6823; C12N 9/226; C12N 15/111; C12Q 1/34; G01N 21/6428; C12N 2310/20; G01N 2021/6432; G01N 2333/922

(57) ABSTRACT

The present disclosure relates to the field of detection technology and discloses a Cas protein and use thereof in detection, wherein the amino acid sequence of the Cas protein is as set forth in SEQ ID NO: 1. The Cas protein obtained in the present disclosure exhibits high cleavage activity, thereby enabling more accurate determination of the amount of low-concentration target substances.

### CROSS-REFERENCE TO RELATED APPLICATIONS

This application claims priority to Chinese Patent Application No. 202411785623.4, filed on Dec. 6, 2024, which is incorporated herein by reference in its entirety.

### TECHNICAL FIELD

### Sequence Listing

This application contains a Sequence Listing which has been submitted in XML format via EFS-Web and is hereby incorporated by reference in its entirety. Said XML copy is named “CAS PROTEIN AND USE THEREOF IN DETECTION CROSS-REFERENCE TO RELATED APPLICATIONS.xml”, created Nov. 12, 2025, and has a size of 8,718 bytes.

The present disclosure relates to the field of detection technology, and particularly relates to a Cas protein and use thereof in detection.

### BACKGROUND

The CRISPR-Cas system originally derives from a natural immune mechanism in bacteria and archaea, serving to defend against invasion by foreign genetic material, such as plasmids and bacteriophages. In recent years, the CRISPR-Cas system, as a revolutionary gene-editing technology, has attracted widespread attention in the field of biological sciences. This technology, characterized by its simplicity and high efficiency, provides a powerful tool for areas including gene editing, gene regulation, and nucleic acid detection. The system achieves recognition and cleavage of specific DNA sequences with high target specificity by utilizing guidance from a Cas nuclease and a crRNA.

CRISPR-Cas detection technology, as an emerging detection method, shows great application potential in fields such as virus detection, disease diagnosis, metal ion detection, and mycotoxin detection. Among these, detection methods based on the trans-cleavage activity of Cas12a have garnered significant attention due to high sensitivity and specificity. However, Cas12a variants currently used in CRISPR-Cas detection still face challenges of insufficient cleavage activity, which limits their widespread adoption in practical applications. Therefore, there is a need to discover or improve existing Cas12a proteins to identify novel Cas12a proteases with better cleavage activity and greater ease of use.

### SUMMARY

To solve the technical problems described above, the present disclosure provides a Cas protein and use thereof in detection. The Cas protein obtained in the present disclosure exhibits high cleavage activity, thereby enabling more accurate determination of the amount of low-concentration target substances.

The present disclosure provides a Cas protein, wherein the amino acid sequence of the Cas protein is as set forth in SEQ ID NO: 1.

The present disclosure also provides a use of the Cas protein in detecting a target molecule.

Further, the target molecule includes one selected from the group consisting of a nucleic acid, a lead metal ion, and a mycotoxin.

Further, the use includes: dispersing a solution of the Cas protein, a solution of a target nucleic acid, a solution of a guide RNA (crRNA), a solution of a nucleic acid probe, nuclease-free water, and a ribonuclease inhibitor in a buffer solution; then adding the target molecule; incubating; and performing fluorescence intensity detection.

Further, the sequence of the nucleic acid probe is TTAATT, wherein an N-terminus of the nucleic acid probe is modified with a fluorescent group, and a C-terminus of the nucleic acid probe is modified with a quenching group.

Further, the fluorescent group is 5′-FAM.

Further, the quenching group is 3′-BHQ1.

Further, the target nucleic acid includes a fragment capable of binding to the guide RNA.

Further, the target nucleic acid includes a fragment specifically capable of binding to the Cas protein.

Further, when the target nucleic acid, the Cas protein, and the guide RNA are combined, the Cas protein is activated and cleaves a single-stranded DNA in the nucleic acid probe.

Further, when the target molecule is a nucleic acid, the target nucleic acid and the target molecule are the same substance, and only the target molecule needs to be added.

Further, when performing detection, one scheme is selected from scheme i to scheme iii:
scheme i: when the target molecule is a nucleic acid, the sequence of the target nucleic acid is consistent with the nucleic acid sequence, and the sequence of the target nucleic acid is as set forth in SEQ ID NO: 6; and the sequence of the guide RNA is as set forth in SEQ ID NO: 7;
scheme ii: when the target molecule is a lead metal ion, the sequence of the target nucleic acid is as set forth in SEQ ID NO: 2; and the sequence of the guide RNA is as set forth in SEQ ID NO: 3; and
scheme iii: when the target molecule is ochratoxin A (OTA), a mycotoxin, the sequence of the target nucleic acid is as set forth in SEQ ID NO: 4; and the sequence of the guide RNA is as set forth in SEQ ID NO: 5.

Further, by volume, a ratio of the solution of the Cas protein, the solution of the guide RNA, the solution of the nucleic acid probe, the nuclease-free water, the buffer solution, the solution of the target nucleic acid, and the ribonuclease inhibitor is 2:2:1:22:2:1:1.

Further, the molar concentration of the solution of the Cas protein is 20 nM.

Further, the preparation method for the solution of the Cas protein specifically includes: dissolving and dispersing the Cas protein in nuclease-free water.

Further, the molar concentration of the solution of the target nucleic acid is 125 nM.

Further, the preparation method for the solution of the target nucleic acid specifically includes: dissolving and dispersing the target nucleic acid in nuclease-free water.

Further, the molar concentration of the solution of the guide RNA is 20 nM.

Further, the preparation method for the solution of the guide RNA specifically includes: dissolving and dispersing the guide RNA in nuclease-free water.

Further, the molar concentration of the solution of the nucleic acid probe is 200 nM.

Further, the preparation method for the solution of the nucleic acid probe specifically includes: dissolving and dispersing the nucleic acid probe in nuclease-free water.

Further, the concentration of the ribonuclease inhibitor is 40 U/μL.

Further, the ribonuclease inhibitor used in the present disclosure is purchased from Sangon Biotech (Shanghai) Co., Ltd., product CAS No.: B600478.

Further, the preparation method for the buffer solution is as follows: 10 mM Tris-HCl (tris(hydroxymethyl)aminomethane hydrochloride), 10 mM magnesium chloride, 10 mM sodium chloride, 100 μg/mL bovine serum albumin (BSA) solution.

Further, the preparation method for the BSA solution specifically includes dispersing the BSA in deionized water.

Further, the temperature for the incubating is 36° C. to 38° C., and the time for the incubating is 14 minutes to 16 minutes.

Further, the fluorescence intensity detection is performed using a microplate reader, with the excitation wavelength of 480 nm to 500 nm and the emission wavelength of 510 nm to 540 nm.

Further, the concentration of the lead metal ion is 0.025 nM to 5 nM.

Further, the concentration of the mycotoxin is 2.5 μg/mL to 1000 μg/mL.

### Technical Effects of Embodiments of the Present Disclosure

1. After adding the target molecule in the present disclosure, when the target nucleic acid, the crRNA, and the Cas protein form a ternary complex, the cleavage activity of the Cas protein is activated, and the fluorescent group on the nucleic acid probe is cleaved by the Cas protein. The Cas protein obtained in the present disclosure exhibits high cleavage activity; therefore, for target substances with low amount, the Cas protein can improve detection accuracy.

2. The Cas protein obtained in the present disclosure exhibits high cleavage activity and can be successfully applied to detect nucleic acids, lead ion amount, and mycotoxins.

### BRIEF DESCRIPTION OF THE DRAWINGS

To illustrate the technical solutions in the embodiments of the present disclosure or in the prior art more clearly, the following provides a brief introduction to the accompanying drawings required for describing the embodiments or the prior art. Obviously, the accompanying drawings in the following description show only some embodiments of the present disclosure. Persons of ordinary skill in the art may obtain other drawings based on these accompanying drawings without creative effort.

FIG. 1 is a diagram showing expression results of the Cas protein provided by an embodiment of the present disclosure.

FIG. 2 is a diagram comparing protein activity of the Cas protein and that of a control group provided by an embodiment of the present disclosure.

FIG. 3 is a standard curve for detection of lead ions using the Cas protein provided by an embodiment of the present disclosure.

FIG. 4 is a diagram showing results of detecting lead ions at known concentrations using the Cas protein provided by an embodiment of the present disclosure.

FIG. 5 is a standard curve for detection of OTA using the Cas protein provided by an embodiment of the present disclosure.

FIG. 6 is a diagram showing results of detecting OTA at known concentrations using the Cas protein provided by an embodiment of the present disclosure.

### DETAILED DESCRIPTION OF THE EMBODIMENTS

To make the objectives, technical solutions, and advantages of the present disclosure clearer, the following describes the technical solutions of the present disclosure clearly and completely. Obviously, the described embodiments are only some, not all, embodiments of the present disclosure. Based on the embodiments in the present disclosure, all other embodiments obtained by persons of ordinary skill in the art without creative effort shall fall within the protection scope of the present disclosure.

In a first aspect, a Cas protein is provided in some embodiments of the present disclosure, wherein the amino acid sequence of the Cas protein is as set forth in SEQ ID NO: 1.

In a second aspect, a use of the Cas protein in detecting a target molecule is further provided in some embodiments of the present disclosure.

In some embodiments, the target molecule includes one selected from the group consisting of a nucleic acid, a lead metal ion, and a mycotoxin.

In some embodiments, the use includes: dispersing a solution of the Cas protein, a solution of a target nucleic acid, a solution of a guide RNA (crRNA), a solution of a nucleic acid probe, nuclease-free water, and a ribonuclease inhibitor in a buffer solution; then adding the target molecule; incubating; and performing fluorescence intensity detection.

In some embodiments, the sequence of the nucleic acid probe is TTAATT, wherein an N-terminus of the nucleic acid probe is modified with a fluorescent group, and a C-terminus of the nucleic acid probe is modified with a quenching group.

In some embodiments, the fluorescent group is 5′-FAM.

In some embodiments, the quenching group is 3′-BHQ1.

In some embodiments, the target nucleic acid includes a fragment capable of binding to the guide RNA.

In some embodiments, the target nucleic acid includes a fragment specifically capable of binding to the Cas protein.

In some embodiments, when the target nucleic acid, the Cas protein, and the guide RNA are combined, the Cas protein is activated and cleaves a single-stranded DNA in the nucleic acid probe.

In some embodiments, when the target molecule is a nucleic acid, the target nucleic acid and the target molecule are the same substance, and only the target molecule needs to be added.

In some embodiments, when performing detection, one scheme is selected from scheme i to scheme iii:
scheme i: when the target molecule is a nucleic acid, the sequence of the target nucleic acid is consistent with the nucleic acid sequence, and the sequence of the target nucleic acid is as set forth in SEQ ID NO: 6; and the sequence of the guide RNA is as set forth in SEQ ID NO: 7;
scheme ii: when the target molecule is a lead metal ion, the sequence of the target nucleic acid is as set forth in SEQ ID NO: 2; and the sequence of the guide RNA is as set forth in SEQ ID NO: 3; and
scheme iii: when the target molecule is OTA, a mycotoxin, the sequence of the target nucleic acid is as set forth in SEQ ID NO: 4; and the sequence of the guide RNA is as set forth in SEQ ID NO: 5.

In some embodiments, by volume, a ratio of the solution of the Cas protein, the solution of the guide RNA, the solution of the nucleic acid probe, the nuclease-free water, the buffer solution, the solution of the target nucleic acid, and the ribonuclease inhibitor is 2:2:1:22:2:1:1.

In some embodiments, the molar concentration of the solution of the Cas protein is 20 nM.

In some embodiments, the preparation method for the solution of the Cas protein specifically includes: dissolving and dispersing the Cas protein in nuclease-free water.

In some embodiments, the molar concentration of the solution of the target nucleic acid is 125 nM.

In some embodiments, the preparation method for the solution of the target nucleic acid specifically includes: dissolving and dispersing the target nucleic acid in nuclease-free water.

In some embodiments, the molar concentration of the solution of the guide RNA is 20 nM.

In some embodiments, the preparation method for the solution of the guide RNA specifically includes: dissolving and dispersing the guide RNA in nuclease-free water.

In some embodiments, the molar concentration of the solution of the nucleic acid probe is 200 nM.

In some embodiments, the preparation method for the solution of the nucleic acid probe specifically includes: dissolving and dispersing the nucleic acid probe in nuclease-free water.

In some embodiments, the concentration of the ribonuclease inhibitor is 40 U/μL.

In some embodiments, the ribonuclease inhibitor used in the present disclosure is purchased from Sangon Biotech (Shanghai) Co., Ltd., product CAS No.: B600478.

In some embodiments, the preparation method for the buffer solution is as follows: 10 mM Tris-HCl, 10 mM magnesium chloride, 10 mM sodium chloride, 100 μg/mL BSA (BSA) solution.

In some embodiments, the preparation method for the BSA solution specifically includes dispersing the BSA in deionized water.

In some embodiments, the temperature for the incubating is 36° C. to 38° C., and the time for the incubating is 14 minutes to 16 minutes.

In some embodiments, the fluorescence intensity detection is performed using a microplate reader, with the excitation wavelength of 480 nm to 500 nm and the emission wavelength of 510 nm to 540 nm.

In some embodiments, the concentration of the lead metal ion is 0.025 nM to 5 nM.

In some embodiments, the concentration of the mycotoxin is 2.5 μg/mL to 1000 μg/mL.

Detailed description is provided below in conjunction with some specific embodiments:

### Embodiment 1

### (1) Construction of the Cas Protein Plasmid:

Using LbCas12a as a seed sequence, a Cas12a protein originating from the bacterium Butyrivibrio_Asp000431815 was discovered through bioinformatics analysis of metagenomic data and was named the Cas protein.

A nucleotide sequence corresponding to the amino acid sequence of the synthesized Cas protein was synthesized. The plasmid containing the synthesized Cas protein was synthesized by Sangon Biotech (Shanghai) Co., Ltd., and the vector for the plasmid was pET-28a(+).

The amino acid sequence of the Cas protein
(SEQ ID NO: 1) is:
MNNNGETKCYGNNLDRFIGLREVNKTLRNELIPTDSTRENIEKNG
IISEDELRAEMGHELKAIMDDYYRNMIEETLSRKCNINWSTLFAA
MDSVARSANKKDADKILEKVQKEKREEIHKLFKEQGNFEKLFKAE
LVSDVLPDFIGKYEGYSDEERISKLGTIKLFKKFMTSFKAYFDNR
KNVFSKENISTSICHRVVNDNAFIFWDNLNAFKKIDKYAHDAVET
IEGEISGKLGEWKISQIYSVDFFGMVIPQSGITFYNEVSGEINKY
MNIYCQKEKQNSRQFKLKRLHKQILCIGETSFEIPEKYESDEEVY
QSVNDFIGMIKNNDIISRLEHLAENVNKYDADKIYINSKSYENVS
KCIGGTWNTIKDCLRLWYNENIRGSGKNKEKKVGKAVEEDKYKSI
AMLNQLLENYSDGDGRIYNVQLYVEHITKIIGDYKPEELVYDANI
RVIEQDEKAEEIKSVLDIYIAILHWIKAFIIEDAVEKDTDFYYEI
EEIFDKVQSIIPLYNMVRNYTTQKPYKQEKIKLKFNTPTLAEGWH
KSKEYSNNAIILQRDGKYYLGIFNAKNKPDKKIIEGCSQKKNKAD
YAKMVYYLLPKPHMNLPHIFIKSKEAVKNYGLSNYIVEGYDKDKH
IKSSNGFDIKYCRDLIDYFKDCISKHPEWKNFNFSFTDTALYNDI
GEFYNEIKRQGYKIEWTYISEEEINRLDENGQIYLFQIYNKDFAE
GSKGTPNLHTLYLKNLFSEDNLKDPVFMLNGNTEVFFRKSSIKKP
VVHKKGSVLVNRNYIDLNGNRITIPEKEYKEIYEYCNGIGNVNLS
ESAMKYYDMAKHYTATMDIVKDYRYTVDKFFIHMPMTINFNAESN
NNINELALNFVAKNDNIHIIGVDRGERNLIYISVIDSKGNIIKQK
SYNVVNDYDYKSKLKEREYDRGDARKNWKRINNIKELKEGYLSQV
VHEIAQLVIQYNAIIAMEDLNYGFKRGRFKVERQVYQKFENMLIN
KLNYLVDKRKNADEAGGVLKGYQLTYIPERLKNMGRQCGIIFYVP
AAYTSKIDPTTGFVNLFNFNAITSQEGRRKFINKFKHIRYNLNMN
LFEFEFDYNDFITHNTQIAKTEWSVYTNGPRIKKERVNGKWNDGY
EADITEVMKQKLSDNGIDYADDGDILTRIAEADDKLVSGIFEIFR
LTVQLRNSKSESCGEDYDRIISPVLNESNRFYDSDEYSGENEILP
VDADANGAYCIALKGLYEVKQIRENWVEGDKLSGDLLKISNADWF
DFIQNKRYE.

### (2) Purification of the Cas Protein:

The Cas protein plasmid obtained in step (1) was transformed into Escherichia coli (E. coli) BL21 (DE3) competent cells. The cells were spread on a commercially purchased E. coli medium containing kanamycin (Kan+) and cultured overnight at 37° C. A single monoclonal colony was then picked and inoculated into 100 mL of an E. coli liquid medium (Kan+). The culture was shaken at 37° C. and 220 rpm until the optical density at 600 nm (OD₆₀₀) reached 0.6, resulting in a Cas protein plasmid bacterial culture. Isopropyl β-D-1-thiogalactopyranoside (IPTG) was added to the Cas protein plasmid bacterial culture to a final concentration of 0.5 mmol/L. Induction was performed at 16° C. and a shaking speed of 200 rpm for 20 hours to obtain an induced bacterial culture.

The induced bacterial culture was centrifuged, and the bacterial pellet was collected. The pellet was resuspended in 20 mL of Buffer A (20 mM Tris-HCl, pH 8.0, 500 mM sodium chloride, 5% glycerol). The resuspended bacterial solution was subjected to ultrasonic disruption at 350 W power, followed by centrifugation for 1 hour. The supernatant was collected for affinity purification. The solution was exchanged and concentrated using a 10K ultrafiltration tube to obtain the purified target protein.

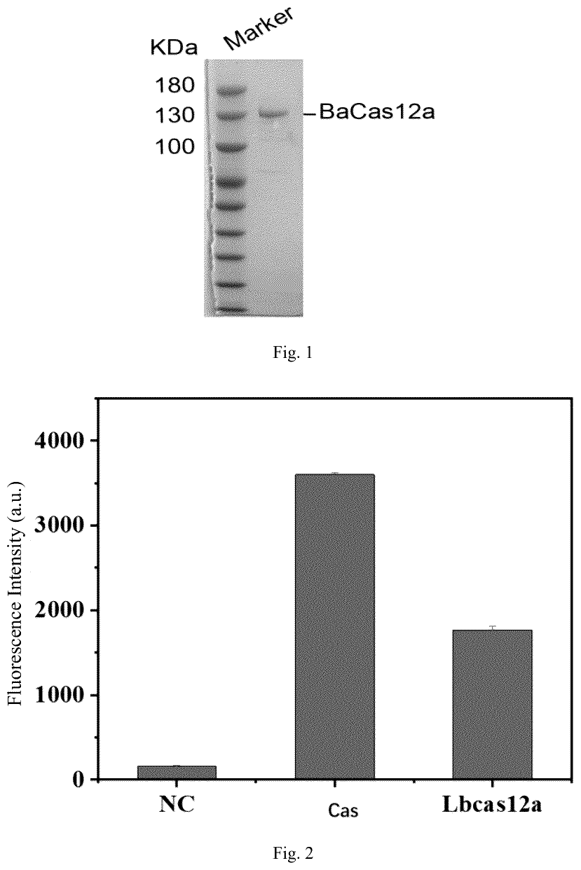

The purified protein was subjected to sodium dodecyl sulfate-polyacrylamide gel electrophoresis (SDS-PAGE), and protein bands were visualized by Coomassie Brilliant Blue staining. The expression results of the obtained Cas protein are shown in FIG. 1. Lane 1 represents the standard protein marker, and Lane 2 represents the Cas protein of the present disclosure (i.e., BaCas12a). The molecular weight of the Cas protein is approximately 130 kDa.

### Embodiment 2: Identification of the Cleavage Activity of the Cas Protein Obtained in Embodiment 1

### (1) Preparation of the Target Molecule:

A target molecule nucleic acid was designed and synthesized by Sangon Biotech (Shanghai) Co., Ltd. The target molecule (target nucleic acid) was obtained by polymerase chain reaction (PCR) amplification at 37° C. for 30 minutes. The sequence is as set forth in SEQ ID NO: 6: GGGGGCACGGGAGGTTGCATCGAGCGGTGG.

### (2) Preparation of the Nucleic Acid Probe:

The structure of the nucleic acid probe was as follows: the N-terminus was modified with a 5′-FAM group, and the C-terminus was modified with a 3′-BHQ1 quenching group. The nucleic acid probe was synthesized by Sangon Biotech (Shanghai) Co., Ltd. The nucleic acid probe sequence was: TTAATT.

### (3) Preparation of the Guide RNA:

A guide sequence was designed based on the characteristics of the target nucleic acid in step (1), as set forth in SEQ ID NO: 7:

The guide RNA sequence is UAAUUUCUACUAAGUGUAGAUAUGCAA CCUCCCGUGCCCCC.

### (4) Identification of Cleavage Activity:

A 30 base pair (bp) nucleic acid sequence (GGGGGCACGGGAGGTTGCAT CGAGCGGTGG) was used as the target site to detect the trans-cleavage activity of the Cas protein against the nucleic acid probe. Specifically, 2 μL of a solution of the Cas protein with a molar concentration of 20 nM was taken. Then, 2 μL of a solution of the guide RNA with a molar concentration of 20 nM, 2 μL of a buffer solution (10 mM T ris-HCl, 10 mM magnesium chloride, 10 mM sodium chloride, 100 μg/mL BSA), 1 μL of a solution of the nucleic acid probe with a molar concentration of 200 nM, 1 μL of a solution of the target nucleic acid with a molar concentration of 125 nM, 1 μL of a ribonuclease inhibitor (concentration 40 U/μL), and 22 μL of nuclease-free water (dd H₂O) were added. The final volume was adjusted to 40 μL with ultrapure water. A sample containing no Cas protein (i.e., Cas protein concentration of 0 nM, labeled as NC) and a sample using LbCas12a protein (i.e., commercially purchased LbCas12a protein at a concentration of 200 nM, labeled as LbCas12a) were used as controls. After incubate ion at 37° C. for 30 minutes, the results were observed under irradiation with a 488 nm ultraviolet lamp. The detection results are shown in FIG. 2.

The results indicate that the sample containing the Cas protein exhibited strong fluorescence, and its fluorescence intensity was far greater than that of the control groups (lacking the Cas protein and the LbCas12a protein). This demonstrates that the Cas protein obtained in the present disclosure possesses superior cleavage activity compared to the LbCas12a protein in the prior art.

### Embodiment 3: Application of the Cas Protein from Embodiment 1 in Detecting Lead Metal Ions

### (1) Preparation of the Target Molecule:

A lead ion-specific GR-5 DNAzyme (as set forth in SEQ ID NO: 2) was used as the target nucleic acid.

The GR-5 DNAzyme sequence, as set forth in SEQ ID NO: 2, is:

ACAGACATCATCTCTGAAGTAGCGCCGCCGTATAGTGAGAAACTC
ACTATrAGGAAGAGATGATGTCTGTAGTT.

### (2) Preparation of the Guide RNA:

A guide sequence was designed based on the characteristics of the target nucleic acid in step (1), as set forth in SEQ ID NO: 3:

UAAUUUCUACUAAGUGUAGAUAACUACAGACAUCAUCUCUUCC.

### (3) Preparation of the Nucleic Acid Probe:

The structure of the nucleic acid probe was as follows: the N-terminus was modified with a 5′-FAM group, and the C-terminus was modified with a 3′-BHQ1 quenching group. The nucleic acid probe was synthesized by Sangon Biotech (Shanghai) Co., Ltd. The nucleic acid probe sequence was: TTAATT.

### (4) Application in Lead Metal Ion Detection:

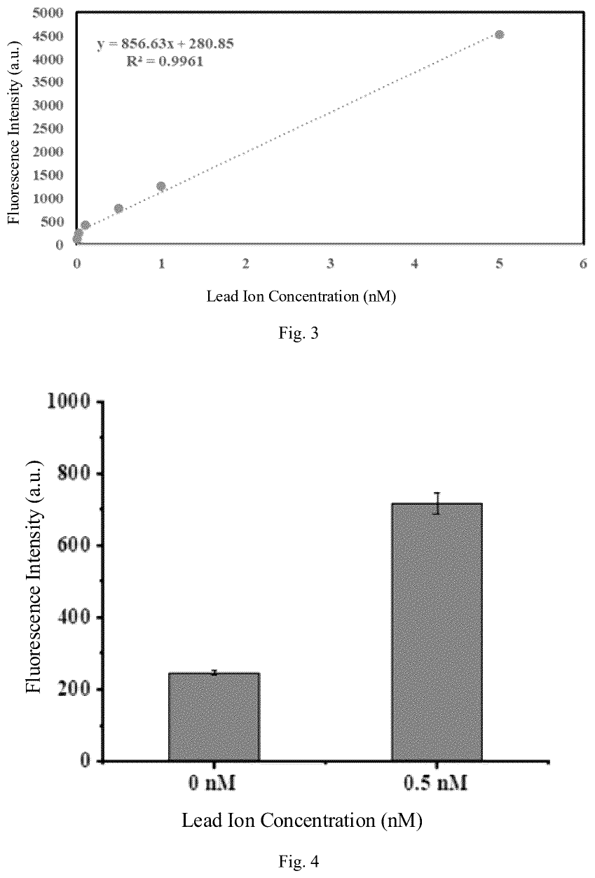

Specifically, 2 μL of a solution of the Cas protein with a molar concentration of 20 nM was taken. Then, 2 μL of a solution of the guide RNA with a molar concentration of 20 nM, 2 μL of a buffer solution (10 mM Tris-HCl, 10 mM magnesium chloride, 10 mM sodium chloride, 100 μg/mL BSA), 1 μL of a solution of the nucleic acid probe with a molar concentration of 200 nM, 1 μL of a solution of the target nucleic acid with a molar concentration of 125 nM, 1 μL of a ribonuclease inhibitor (concentration 40 U/μL), and 22 μL of nuclease-free water (dd H₂O) were added. The final volume was adjusted to 40 μL with ultrapure water. Solutions with standard lead ion concentrations of 0 nM, 0.02 nM, 0.1 nM, 0.5 nM, 1 nM, and 5 nM were detected separately. After incubation at 37° C. for 30 minutes, the fluorescence intensity was recorded at an excitation wavelength of 485 nm and an emission wavelength of 530 nm. A standard curve relating lead ion concentration to fluorescence intensity was established (as shown in FIG. 3). The standard curve was determined to be y=856.63x+280.85, with R²=0.996, and the calculated detection limit for lead ions was 0.025 nM. The method of the present disclosure was then used to detect a solution with a known lead ion concentration of 0.5 nM. The detected fluorescence intensity was 709 a.u. (as shown in FIG. 4). The fluorescence intensity from FIG. 4 was substituted into the standard curve. The results showed that for the sample with a known lead ion concentration of 0.5 nM, the calculated concentration was 0.49 nM. This demonstrates that the Cas protein obtained in the present disclosure exhibits higher activity and can accurately detect solutions containing low concentrations of lead ions.

### Embodiment 4: Application of the Cas Protein from Embodiment 1 in Detecting Mycotoxins

### (1) Preparation of the Target Molecule:

A mycotoxin was used as the target molecule, and a corresponding aptamer DNA was designed and constructed as the target nucleic acid. Taking OTA (OTA) as an example, the sequence of the target nucleic acid is as set forth in SEQ ID NO: 4:

5′-TTTTTTATCGACCGATGCTCCATAG-3′.

### (2) Preparation of the Guide RNA:

A guide RNA sequence was designed based on the characteristics of the target molecule, as set forth in SEQ ID NO: 5:

UAAUUUCUACUAAGUGUAGAUCUAUGGAGCAUCGGUCGAUAAAA.

### (3) Preparation of the Nucleic Acid Probe:

The structure of the nucleic acid probe was as follows: the N-terminus was modified with a 5′-FAM group, and the C-terminus was modified with a 3′-BHQ1 quenching group. The nucleic acid probe was synthesized by Sangon Biotech (Shanghai) Co., Ltd. The nucleic acid probe sequence was: TTAATT.

### (4) Application in Mycotoxin Detection:

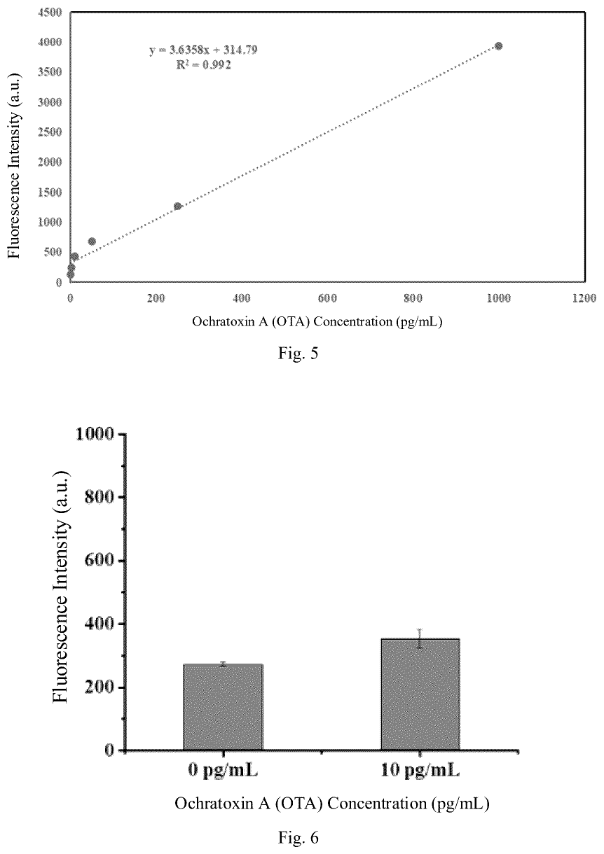

2 μL of a solution of the Cas protein with a molar concentration of 20 nM was taken. Then, 2 μL of a solution of the guide RNA with a molar concentration of 20 nM, 2 μL of a buffer solution (10 mM Tris-HCl, 10 mM magnesium chloride, 10 mM sodium chloride, 100 μg/mL BSA), 1 μL of a solution of the nucleic acid probe with a molar concentration of 200 nM, 1 μL of a solution of the target nucleic acid with a molar concentration of 125 nM, 1 μL of a ribonuclease inhibitor (concentration 40 U/μL), and 22 μL of nuclease-free water (dd H₂O) were added. The final volume was adjusted to 40 μL with ultrapure water. Standard OTA solutions with concentrations of 0 μg/mL, 2 μg/mL, 10 μg/mL, 50 μg/mL, 250 μg/mL, and 1000 μg/mL were separately detected. After incubation at 37° C. for 30 minutes, the fluorescence intensity was recorded at an excitation wavelength of 485 nm and an emission wavelength of 530 nm. A suitable standard curve relating OTA concentration to fluorescence intensity was established (as shown in FIG. 5). The standard curve was determined to be y=3.6358x+314.79, with R²=0.992, and the calculated detection limit for OTA was 2.5 μg/mL. The method of the present disclosure was then used to detect a solution with a known OTA concentration of 10 μg/mL. The detected fluorescence intensity was 351 a.u. (as shown in FIG. 6). The fluorescence intensity from FIG. 6 was substituted into the standard curve. The results showed that for the sample with a known OTA concentration of 10 μg/mL, the calculated concentration was 9.95 μg/mL. This demonstrates that the Cas protein obtained in the present disclosure exhibits higher activity.

It is to be noted that the terminology used in the present disclosure is for the purpose of describing particular embodiments only and is not intended to limit the scope of the present disclosure. As used in the specification of the present disclosure, unless the context clearly indicates otherwise, the articles “a”, “an”, and/or “the” are intended to mean that one or more of the elements are present. The term “comprising”, “including”, or any variation thereof, is intended to encompass a non-exclusive inclusion, such that a process, method, or apparatus that comprises a list of elements does not include only those elements but may include other elements not expressly listed or inherent to such process, method, or apparatus. Without further limitation, an element defined by the phrase “comprising a . . . ” does not exclude the presence of additional identical elements in the process, method, or apparatus that comprises the element.

It is further to be noted that terms indicating orientation or positional relationships, such as “center”, “upper”, “lower”, “left”, “right”, “vertical”, “horizontal”, “inner”, “outer”, and the like, are based on the orientation or positional relationships shown in the accompanying drawings. These terms are used merely for the purpose of facilitating the description of the present disclosure and simplifying the description, rather than indicating or implying that the referred apparatus or element must have a specific orientation, or be constructed and operated in a specific orientation. Therefore, these terms are not to be construed as limiting the present disclosure. Unless otherwise expressly specified and defined, terms such as “mounted”, “connected with”, and “connected to” are to be construed broadly. For example, a connection may be a fixed connection, a detachable connection, or an integral connection; a connection may be a mechanical connection, an electrical connection; and a connection may be a direct connection, an indirect connection through an intermediate medium, or an internal communication between two elements. The specific meanings of the above-mentioned terms in the present disclosure can be understood by those of ordinary skill in the art according to specific circumstances.

Finally, it should be finally noted that the above embodiments are merely intended to illustrate the technical solutions of the present disclosure, but not to construe them as limitations. Although the present disclosure has been described in detail with reference to the foregoing embodiments, persons of ordinary skill in the art should understand that they may still make modifications to the technical solutions described in the foregoing embodiments, or make equivalent substitutions for some or all of the technical features therein. However, these modifications or substitutions do not make the essence of the corresponding technical solutions depart from the scope of the technical solutions of the embodiments of the present disclosure.

What is claimed is:

1. A Cas protein, wherein the amino acid sequence of the Cas protein is as set forth in SEQ ID NO: 1.

2. Use of the Cas protein according to claim 1 in detecting a target molecule; wherein the use is for a non-disease diagnostic purpose and/or a non-therapeutic purpose.

3. The use according to claim 2, wherein the target molecule comprises one selected from the group consisting of a nucleic acid, a lead metal ion, and a mycotoxin.

4. The use according to claim 3, wherein the use comprises: dispersing a solution of the Cas protein, a solution of a target nucleic acid, a solution of a guide RNA, a solution of a nucleic acid probe, nuclease-free water, and a ribonuclease inhibitor in a buffer solution; then adding the target molecule; incubating; and performing fluorescence intensity detection.

5. The use according to claim 4, wherein the sequence of the nucleic acid probe is TTAATT, an N-terminus of the nucleic acid probe is modified with a fluorescent group, a C-terminus of the nucleic acid probe is modified with a quenching group; wherein the fluorescent group is 5′-FAM; and the quenching group is 3′-BHQ1.

6. The use according to claim 4, wherein the target nucleic acid comprises a fragment capable of binding to the guide RNA;
the target nucleic acid comprises a fragment specifically capable of binding to the Cas protein; and
when the target nucleic acid, the Cas protein, and the guide RNA are combined, the Cas protein is activated and cleaves a single-stranded DNA in the nucleic acid probe.

7. The use according to claim 4, wherein, when performing detection, one scheme is selected from scheme i to scheme iii:
scheme i: when the target molecule is a nucleic acid, the sequence of the target nucleic acid is consistent with the nucleic acid sequence, and the sequence of the target nucleic acid is as set forth in SEQ ID NO: 6; and the sequence of the guide RNA is as set forth in SEQ ID NO: 7;
scheme ii: when the target molecule is a lead metal ion, the sequence of the target nucleic acid is as set forth in SEQ ID NO: 2; and the sequence of the guide RNA is as set forth in SEQ ID NO: 3; and
scheme iii: when the target molecule is ochratoxin A (OTA), a mycotoxin, the sequence of the target nucleic acid is as set forth in SEQ ID NO: 4; and the sequence of the guide RNA is as set forth in SEQ ID NO: 5.

8. The use according to claim 4, wherein, by volume, a ratio of the solution of the Cas protein, the solution of the guide RNA, the solution of the nucleic acid probe, the nuclease-free water, the buffer solution, the solution of the target nucleic acid, and the ribonuclease inhibitor is 2:2:1:22:2:1:1;
wherein the molar concentration of the solution of the Cas protein is 20 nM;
wherein the molar concentration of the solution of the target nucleic acid is 125 nM;
wherein the molar concentration of the solution of the guide RNA is 20 nM;
wherein the molar concentration of the solution of the nucleic acid probe is 200 nM; and
wherein the concentration of the ribonuclease inhibitor is 40 U/μL.

9. The use according to claim 4, wherein the temperature for the incubating is 36° C. to 38° C., and the time for the incubating is 14 minutes to 16 minutes; and
the fluorescence intensity detection is performed using a microplate reader, with the excitation wavelength of 480 nm to 500 nm and the emission wavelength of 510 nm to 540 nm.

10. The use according to claim 3, wherein the concentration of the lead metal ion is 0.025 nM to 5 nM; and
the concentration of the mycotoxin is 2.5 μg/mL to 1000 μg/mL.
